(19) United States
(12) Patent Application Publication
(10) Pub. No.: US 2025/0082708 A1
(43) Pub. Date: Mar. 13, 2025

(54) HERBAL COMPOSITION FOR USE IN THE TREATMENT OR PREVENTION OF POSTPARTUM DEPRESSION

(71) Applicant: CRISTALFARMA S.R.L., Milano (IT)

(72) Inventors: Maria Cristina Del Bono, Milano MI (IT); Francesco Bonomo, Milano MI (IT)

(73) Assignee: CRISTALFARMA S.R.L., Milano MI (IT)

(21) Appl. No.: 18/868,078

(22) PCT Filed: May 24, 2023
(86) PCT No.: PCT/IB2023/055338

§ 371 Date: Nov. 21, 2024

(30) Foreign Application Priority Data
May 25, 2022 (IT) 102022000010973

(51) Int. Cl. A61K 36/53; A61K 31/593; A61K 36/81; A61P 25/24

(52) U.S. Cl. CPC A61K 36/53; A61K 31/593; A61K 36/81; A61P 25/24

(57) ABSTRACT

The present invention concerns a composition comprising, as active ingredients: —dry extract of Withania somnifera, —dry extract of Melissa officinalis, —microencapsulated essential oil of Lavandula angustifolia, and —optionally, Vitamin D3 for use in the prevention and/or in the treatment of postpartum depression (PPD).

### FIELD OF THE INVENTION

The composition of the present invention falls within the technical field of compositions or combinations of active ingredients useful for the treatment of postpartum depression.

### STATE OF THE ART

Following childbirth, a type of major depressive disorder—known as postpartum depression—affects about 8-12% of new mothers. Constituting one of the most common medical complications during the postnatal period, postpartum depression (PPD) comprises major and minor depressive episodes that occur during pregnancy or in the first 12 months after delivery, affecting one in seven women. PPD can have devastating effects on women, children and families; maternal suicide outweighs bleeding and hypertensive disorders as a cause of maternal mortality.

Compared to depression that occurs in periods other than the postnatal one, PPD is currently little studied, although in the most recent scientific literature started to discuss about the definition of this disorder and, more specifically, about the difference between PPD and depression that occurs in periods other than the postnatal one.

Di Florio et Al. (Di Florio A, Meltzer-Brody S., “Is Postpartum Depression a Distinct Disorder?”. Curr Psychiatry Rep. 2015 October; 17(10):76) highlight the reasons why postpartum depression (PPD) should be considered as a pathology distinct from depression. In particular, the reasons that support a distinction between the two pathologies are: 1) Guidelines: there are guidelines (such as the NICE and SIGN guidelines) specific to postpartum depression, as the identification and the treatment of women with PPD requires specific considerations, which usually do not apply to depression outside of the perinatal period. In addition, the therapeutic needs of women with PPD differ from those of the women with normal depression, often requiring a multidisciplinary approach (physicians need to consider the implications of the psychotropic medications during breastfeeding and recognize and treat the mother in the context of the relationship with the child). 2) Prognosis and outcomes: some epidemiological and clinical studies have suggested an association between bipolar diathesis and PPD; the evidence of a link between childbirth and bipolar disorder therefore leads physicians to be particularly careful in the assessment, treatment and follow-up of the women with PPD, as they have an increased risk of bipolar diathesis. This characteristic also differentiates women with PPD from women with depression outside of the perinatal period. 3) Genetic factors: studies have shown that there are genetic differences between women with PPD and women with depression outside of the perinatal period. It is therefore plausible that the two pathologies require different treatments and clinical interventions and this reinforces the idea of considering the two pathologies as distinct pathologies. 4) Epidemiology: many epidemiological and clinical studies have shown that depression is more common after childbirth than at other times in a woman's life, suggesting an aetiological link precisely with this particular moment in a woman's life.

Batt et al. (Melissa M Batt et al. “Is Postpartum Depression Different From Depression Occurring Outside of the Perinatal Period?A Review of the Evidence”. Focus (Am Psychiatr Publ) 2020 April; 18(2): 106-119) highlight the similarities and the differences between PPD and depression outside the postnatal period. In particular, it is pointed out that the differentiation between the two types of depression is very evident if one considers as postpartum the period that goes up to 8 weeks after the birth of the child because, in this time frame, the severity of the symptoms, the hormone level, the heredity, the epigenetic data and the response to treatment are different from those of the postpartum period after 8 weeks, where they are more similar to those of depression that occurs outside the perinatal period.

With regard also to treatment, it has been seen that there are probable benefits in the treatment of early PPD compared to late PPD, which instead responds to the treatment as if it were a normal depression.

According to NICE Guidelines (Antenatal and postnatal mental health: clinical management and service guidance. www.nice.org.uk/guidance/cg192), the management of PPD differs from other depressions because of the particular nature of this phase of a woman's life and also because of the potential impact that this type of depression can have not only on the woman but also on the child. This publication also highlights the fact that there are no approved psychotropic drugs for postpartum depression in Europe; it is therefore the attending physician who must take on the responsibility of prescribing a psychotropic drug in this very delicate phase of a woman's life. Psychotropic drugs, in fact, cannot be used during breastfeeding and they can alter a woman's ability to care for her baby (e.g., sedatives).

Comparative studies were also conducted aimed at comparing the symptomatology and the biological responses of patients suffering from normal depression and patients suffering from PPD or in conditions simulating PPD.

A retrospective study compared the symptoms and the responses to the treatments of 26 women with PPD and 25 women with normal depression. It was noted that:
Women with PPD experienced a stronger state of anxiety and more severe depression than women with normal depression;
In women with PPD, the response time to pharmacotherapy was longer than that of women with normal depression (6 weeks vs 3 weeks);
When drug therapy response was achieved, 60% of women with PPD were treated not with a single depressive agent, but with multiple depressive agents (compared to 4% of women with normal depression).

The authors therefore conclude that there are evident differences between PPD and normal depression: women with PPD, in fact, have more evident anxious characteristics, take longer to respond to the antidepressant drugs and require the use of more antidepressant drugs to achieve the response to treatment (Hendrick V et al., “Postpartum and nonpostpartum depression: differences in presentation and response to pharmacologic treatment”. Depress Anxiety 2000; 11(2):66-72).

O'Brien et al., (O'Brien S et al., “Is postnatal depression a distinct subtype of major depressive disorder? An exploratory study”. Arch Womens Ment Health. 2021 April; 24(2):329-333) conducted an exploratory study to test whether women with a history of PPD have specific differences in brain activation compared to women with normal depression. This test was done by subjecting the women enrolled to a functional magnetic resonance imaging (fMRI) scan that measured brain activation at the level of the amygdala, a nuclear complex located in the temporal lobe of the brain that manages emotions.

This study was based on the results of two previous studies: the first study (Groenewold et al. 2013) observed that women with a prior history of depression (not PPD), who were subjected to fMRI, experienced increased activation of the amygdala when subjected to negative facial expressions. The second study (Moses-Kolko et al. 2010) observed instead that women with PPD experienced a reduced activation of the amygdala when subjected to negative facial expressions.

The aim of O'Brien et Al.'s study is to make a direct comparison between women with a history of normal depression and women with a history of PPD (vs control). It has also been shown that a key factor for the development of PPD is the abrupt decrease in sex hormones that occurs after childbirth; women who tend towards PPD are in fact particularly sensitive to these changes. This is the reason why, in the O'Brien et al.'s exploratory study, measurements were made in the late luteal phase of the menstrual cycle, since the abrupt decrease in the concentration of estradiol and plasma progesterone in this phase resembles, in a resized way, the postnatal hormonal environment. In this way, it was possible to evaluate how brain activation changes in women with PPD and in women with normal depression, in a situation that simulates the hormonal state of the postpartum.

From the O'Brien et al.'s study, it emerged that women with a history of PPD had a reduced brain activity in the amygdala compared to women with a previous history of normal depression. It has therefore been hypothesized that in women who develop PPD there is a different brain activation compared to women who develop normal depression, which supports the idea that the two pathologies are distinct from each other.

### Problems of the Background Art

Although the existence of PPD is known to many, little is known about the pharmacological management of the patient who, sometimes, is treated with the same therapies used to treat major depressive disorders, which occur in periods other than the postnatal one.

This strategy is inadequate, since there is a need to avoid therapies that can harm the child directly—by exposure to drug therapy through breastfeeding—or indirectly—because the mother's mood and the resources can be altered by the current drug therapy.

As highlighted by the NICE Guidelines, there are currently no approved psychotropic drugs for postpartum depression in Europe, placing the attending physician in the position of having to evaluate, on a case-by-case basis, which therapy to prescribe to treat the mother and minimize the impact that she can also have on the child.

PPD should be considered as a separate pathology, precisely because of this difference in clinical practice, as well as because of a symptomatic picture that is different from that of normal depression: compared to mothers without depression, mothers with PPD tend to have a lower quality of interaction with their children, consisting of a greater disconnection from the offspring and a less positive affection. These aspects of PPD impact on maternal behaviour and on the gratification of the mother's role; as well as on the child, in ensuring an optimal evolutionary outcome. PPD is indeed associated with deficits in cognitive and socio-emotional development among children, and evidence suggests that the quality of mother-child interaction mediates the outcomes of such development (Melissa M Batt et al. “Is Postpartum Depression Different From Depression Occurring Outside of the Perinatal Period? A Review of the Evidence”. Focus (Am Psychiatr Publ) 2020 April; 18(2):106-119).

Hence the need arises to have a natural product available that can be used in the postpartum period and during breastfeeding without problems.

At the state of the art, there are natural remedies useful for the treatment of anxiety disorders or disorders attributable to depressive forms; at the state of the art, however, none of these has found application in the prevention and/or to the treatment of postpartum depression.

EP 1796702 describes the use of lavender oil (Lavandula angustifolia; [0009]) for the treatment of patients suffering from somatization disorders and post-traumatic stress syndrome (PTSD; paragraph [0011]). The daily dosage to be administered is comprised between 10 mg and 2 g, preferably between 50 and 100 mg (par. [0017]).

WO 2011/098394 describes a composition for the treatment and the prophylaxis of nervous and sleep disorders, comprising extracts/tinctures of the following plants: Lavandula angustifolia (lavender), Humulus lupulus (hops), Melissa officinalis (Melissa), Passiflora incarnata (passionflower) and Valeriana officinalis (valerian) (page 1, paragraphs 12-13). Nerve disorders are defined in the document as characterized by increased excitability, irritability, sleep disorders, insomnia, resignation, depressive detuning, decreased libido, exhaustion, anxiety, nervousness, climacteric disorders, shortness of breath, headache, migraine, muscle contractions or convulsions, heart problems (irregular heartbeat, heart pain), stomach, bowel or bladder cramps (page 2, paragraph 3).

WO 2007/014334 describes a pharmaceutical, veterinary or nutraceutical product comprising the extract of Withania somnifera in a concentration comprised between 0.05% and 99% by weight. The composition may also include other active ingredients, such as antioxidants, vitamins, minerals or plant extracts (page 6, lines 1-3). The composition is intended for the treatment of stress disorders characterized by a symptomatology comprising insomnia, palpitations, sweating, fatigue, irritability, feeling of inevitability/imminent fate (claim 10).

WO 2020/144591 describes Withania somnifera delivery systems and in particular a solid dosage form, comprising a core, containing a solid particle (preferably consisting of saccharides or derivatives; page 6, lines 9-11) and a layer of Withania extract covering the particle; a layer disposed above the core, functional to protect the Withania from attack by the stomach gastric juices (page 2, lines 19-25). The composition has several biological activities: immunomodulatory, reduction of depressive states or anxiety, improvement of sleep disorders, improvement of testosterone level in men and duration of sexual performance. The composition, intended for the treatment of depression, anxiety, sleep disorders, is to be administered at a dosage comprised between 10 mg and 2000 mg in subjects with these disorders (page 6, line 27—page 7, line 6).

### SUMMARY OF THE INVENTION

The Applicant has now identified a composition useful for the prevention and the treatment of postpartum depression, which solves the problems of the known art.

The composition comprises, as active ingredients:
dry extract of Withania somnifera,
dry extract of Melissa officinalis, and
microencapsulated essential oil of Lavandula angustifolia.

According to a particularly preferred embodiment, the composition may further comprise Vitamin D3.

### Advantages of the Invention

The composition developed by the Applicant is advantageous because:
it is a natural product, safe for use in pregnant or lactating women;
it constitutes a treatment dedicated to postpartum depression, thus offering patients and the physician a specific therapeutic proposal for PPD, which currently does not exist;
it can be taken alone or as an adjuvant to classic antidepressant or anxiolytic drug therapy, allowing to reduce the number of traditional drug treatments. As indicated in the literature, in fact, 60% of women with PPD are treated with the use of more depressive agents, compared to 4% of women with normal depression (Hendrick V et al., “Postpartum and nonpostpartum depression: differences in presentation and response to pharmacologic treatment”. Depress Anxiety 2000; 11(2):66-72).

### DESCRIPTION OF THE DRAWINGS

FIG. 1: Bar graph depicting the sum of the mean scores recorded for each of the 10 items of the EPDS questionnaire. The woman defines herself as suffering from PPD when the score is >12.5.

FIG. 2A-2B: Bar graphs depicting the mean scores recorded for each of the 10 items of the EPDS questionnaire.

### DETAILED DESCRIPTION OF THE INVENTION

### Composition.

As anticipated in the summary of the invention, the composition subject to protection comprises, as active ingredients,
dry extract of Withania somnifera,
dry extract of Melissa officinalis, and
microencapsulated essential oil of Lavandula angustifolia.

The dry extract of Withania somnifera, the dry extract of Melissa officinalis, and the microencapsulated essential oil of Lavandula angustifolia are also mentioned in the text as “herbal” or as “herbal active ingredients”, since they are active ingredients of plant origin.

According to a preferred embodiment, the dry extract of Withania somnifera, the dry extract of Melissa officinalis, and the microencapsulated essential oil of Lavandula angustifolia constitute the only herbal active ingredients of the composition subject-matter of the invention.

Although the Applicant does not currently have supporting experimental evidence, he considers that the combination of dry extract of Withania somnifera, dry extract of Melissa officinalis, and microencapsulated essential oil of Lavandula angustifolia can be synergistic in preventing and treating postpartum depression, as well as in the prevention and treatment of the symptomatology associated therewith.

Preferably, the composition does not contain: Hawthorn, such as for example Crataegus pinnatifida or Crataegus Oxyacantha, or extracts/derivatives thereof; Lavandula officinalis or extracts/derivatives thereof; Humulus lupulus or extracts/derivatives thereof, Valeriana officinalis or extracts/derivatives thereof, Cinnamomum cassia or extracts/derivatives thereof, fruits of the genus Citrus, such as Citrus bergamia, Citrus Union, Citrus paradisi, or extracts/derivatives thereof.

Withania somnifera (Ashwagandha) has been used in medicine since Ayurveda, the ancient Indian system of medicine. Ashwagandha has been used as an aphrodisiac, liver tonic, anti-inflammatory agent, astringent, and to treat bronchitis, asthma, ulcers, emaciation, insomnia and senile dementia. More recently, W. somnifera has also proven useful for the treatment of anxiety, cognitive and neurological disorders, inflammations and Parkinson's disease (Singh, G & Sharma, P. K. & Dudhe, Rupesh & Singh, Sukhdev. (2009). Biological activities of Withania somnifera. Ann Biol Res. 1).

For the purposes of the invention, the dry extract of W. somnifera is preferably an extract of roots of the plant. Still, the extract of W. somnifera is preferably characterized by a thiol in total withanolides≥5% by weight, on the total weight of the extract; and a titre in withaferin A<0.10% by weight, on the total weight of the extract.

M. officinalis is a medicinal plant that has long been used in several ethnomedical approaches especially in the traditional medicine for the treatment of several diseases (health syndromes including neurological, central nervous system, cardiovascular ones); as an antimicrobial, antidepressant and as a memory enhancer for subjects suffering from dementia (Javad Sharifi-Rad et al “Phytochemical Constituents, Biological Activities, and Health-Promoting Effects of the Melissa officinalis”, Oxidative Medicine and Cellular Longevity, vol. 2021, Article ID 6584693, 20 pages, 2021. https://doi.org/10.1155/2021/6584693).

For the purposes of the invention, the dry extract of M. officinalis is preferably an extract of leaves of the plant, preferably characterized by an extract/drug ratio comprised between 1:4 and 1:6. Still, the dry extract of M. officinalis is preferably characterized by a thiol in rosmarinic acid≥12% by weight, on the total weight of the extract.

The lavender essential oil is used in a wide range of products, both for cosmetic and therapeutic application; oils from a variety of lavender species have been shown to have multiple biological activities. L. angustifolia is well known and appreciated by the cosmetic, food and pharmaceutical industry as an aromatic and medicinal herb. The lavender essential oil is widely sold as over-the-counter phytotherapy for the treatment of depression, anxiety, and stress. International organizations, including the World Health Organization (WHO), the European Medicines Agency (EMA) and the European Scientific Cooperative on Phytotherapy (ESCOP), recognize lavender as an approved remedy for relieving anxiety, stress and restlessness.

In the purposes of the invention, the lavender essential oil is preferably an extract of flowers of L. angustifolia.

The oil is in microencapsulated form, wherein by microencapsulation is meant a technique of delivery in which the active ingredient is included in a particle structure provided with an exterior suitable to maintain the biological, functional and physico-chemical properties of the core materials. The microencapsulation of essential oils is known to the person skilled in the art and is commonly achievable with different methodologies such as, for example, emulsification, spray-drying, coaxial electrospraying, freeze-drying, coacervation, in situ polymerization, melt extrusion, supercritical fluid technology and fluid bed coating. Coacervation and spray drying are the most commonly used techniques for the microencapsulation of essential oils.

According to the preferred embodiment, the essential oil microcapsules of L. angustifolia contain about between 80% and 90% by weight of essential oil, preferably between about 85% and 90% of essential oil. Preferably, the essential oil microcapsules of L. angustifolia are characterized by particle sizes comprised between about 100 m and 600 m (more precisely 80% of the particle population of a microencapsulated essential oil sample of L. angustifolia has particle sizes comprised between 100 m and 600 m, determined by Dynamic Light Scattering).

Still preferably, the essential oil of L. angustifolia included in the microcapsules is characterized by a titre in linalool comprised between about 30% and 40% by weight, on the total weight of the oil; in linalool acetate comprised between about 30% and 40% by weight, on the total weight of the oil; in linalyl acetate comprised between about 30% and 40% by weight, on the total weight of the oil.

Preferably, the dry extract of W. somnifera is contained in the composition in a concentration comprised between about 30% and 40%, preferably between about 32% and 38%, preferably between about 35% and 38% by weight, on the total weight of the composition.

Preferably, the dry extract of M. officinalis is contained in the composition in a concentration comprised between about 20% and 30%, preferably between about 22% and 27%, preferably between about 23% and 26% by weight, on the total weight of the composition.

Preferably, the microencapsulated essential oil of L. angustifolia is contained in the composition in a concentration comprised between about 20% and 30%, preferably between about 22% and 27%, between about 23% and 26% by weight, on the total weight of the composition.

According to a preferred embodiment, an administration unit of the composition comprises:
dry extract of Withania somnifera in a concentration comprised between 30% and 40% (w/w), and preferably having a titre in withanolides≥5%;
dry extract of Melissa officinalis, in a concentration comprised between 20% and 30% (w/w), and preferably having a titre in rosmarinic acid≥12%; and
microencapsulated essential oil of Lavandula angustifolia, in a concentration comprised between 20% and 30% (w/w), wherein the essential oil is preferably characterized by a titre in linalool comprised between 30% and 40%.

According to a particularly preferred embodiment, the composition comprises, as a further (non-herbal) active ingredient, vitamin D3.

Preferably, an administration unit of the composition comprises Vitamin D3 in a concentration comprised between 3% and 8% by weight, on the total weight of the composition.

Note that, preferably, Vitamin D3 is the only vitamin present in the composition of the invention.

The advantage deriving from the association of Vitamin D3 with the previously mentioned and described active ingredients derives from the fact that the scientific literature shows a direct correlation between vitamin D deficiency and the onset or progression of postpartum depression (Fallah M et al., “Is Vitamin D Status Associated with Depression, Anxiety and Sleep Quality in Pregnancy: A Systematic Review.” Adv Biomed Res. 2020 Jul. 27; 9:32; Murphy P K et al. (Murphy P K et al., “An exploratory study of postpartum depression and vitamin d.” J Am Psychiatr Nurses Assoc 2010; 16:170 7; Gur E B et al., “Mid pregnancy vitamin D levels and postpartum depression.” Eur J Obstet Gynecol Reprod Biol 2014; 179:110 6; Robinson M et al., “Low maternal serum vitamin D during pregnancy and the risk for postpartum depression symptoms.” Arch Womens Ment Health 2014; 17:213 9; Fu C W et al., “Association between serum 25 hydroxyvitamin D levels measured 24 hours after delivery and postpartum depression.” BJOG 2015; 122:1688 94.)

Particularly interesting is the study by Amini et al. (Amini S et al., “The effect of vitamin D and calcium supplementation on inflammatory biomarkers, estradiol levels and severity of symptoms in women with postpartum depression: A randomized double blind clinical trial.” Nutr Neurosci 2022; 25(1):22-32.) in which the effect of vitamin D and calcium on the severity of symptoms and inflammatory biomarkers related to PPD was studied. In the study (randomized double-blind) 27 women were enrolled, divided into 3 groups (ratio 1:1:1):
first group: vitamin D+500 mg of calcium
second group: vitamin D+calcium placebo
third group: vitamin D placebo+calcium placebo (control).

The treatment lasted 8 weeks, at the end of which the vitamin D level and the EPDS score were compared with respect to the baseline. It was seen that the EPDS questionnaire score had a greater reduction with respect to the baseline in the group treated with (vitamin D+calcium) and in the group treated with (vitamin D+calcium placebo) with respect to the control group treated with placebo only; moreover, the effect of vitamin D on the EPDS score was greater in the group treated only with vitamin D without the addition of calcium. The authors therefore concluded that vitamin D could be effective in improving the clinical symptoms of PPD.

According to a preferred embodiment, an administration unit of the composition comprises:
dry extract of Withania somnifera in a concentration comprised between 30% and 40% (w/w), and preferably having a titre in withanolides≥5%;
dry extract of Melissa officinalis, in a concentration comprised between 20% and 30% (w/w), and preferably having a rosmarinic acid titre of ≥12%;
microencapsulated essential oil of Lavandula angustifolia, in a concentration comprised between 20% and 30% (w/w), wherein the essential oil is preferably characterized by a titre in linalool between 30% and 40%; and
Vitamin D3 in a concentration comprised between 3% and 8% (w/w).

Preferably, the composition of the invention also comprises suitable excipients and/or diluents, useful for the formulation of a product intended for oral intake. By way of example, the excipients and/or diluents may be chosen from anti-aggregating agents (such as microcrystalline cellulose), bulking agents, anti-packing agents or flow agents (such as silica dioxide), emulsifying agents, glidating agents (such as the magnesium salt of fatty acids), stabilizing agents, dyes, flavours.

According to a preferred embodiment of the invention, the dry extract of Withania somnifera, the dry extract of Melissa officinalis, the microencapsulated essential oil of Lavandula angustifolia and Vitamin D3 constitute the only active ingredients of the composition subject-matter of the invention.

### Forms of Administration of the Composition.

Preferably, the composition of the invention is intended for oral administration.

Preferably, the composition of the invention is characterized by a daily dosage of 1 or 2 administration units.

Preferably, the composition, i.e. the administration unit of the composition, is in solid or liquid form, preferably solid.

Still preferably, the composition, i.e. the administration unit of the composition, is in solid tablet, capsule, or powder/granulate form.

Note that, preferably, when the composition is in powder/granulate form, the powder/granulate is to be dissolved in water, before administration; or by oral absorption.

According to a preferred embodiment, the composition of the invention is a food supplement.

By food supplement is meant a formulation falling within the definition underlying the Directive 2002/46/EC as amended. In this norm, food supplements are defined precisely as: “foodstuffs whose purpose is to supplement the common diet and which are a concentrated source of nutrients, such as vitamins and minerals, or other substances with a nutritional or physiological effect, in particular, but not exclusively, amino acids, essential fatty acids, fibres and extracts of plant origin, both mono- and multi-compounds, in pre-dosed forms”.

### Medical Use.

As highlighted in the summary of the invention, the disclosed composition is intended for the prevention and the treatment of postpartum depression (PPD).

According to a preferred embodiment, the composition is used as an adjuvant of drug therapies. Note that the treatment of PPD can vary depending on the subject and generally includes psychological interventions (psychotherapy), and/or the use of neuro-active drugs (sedatives, anxiolytics, mood regulators, antidepressants) with all the appropriate precautions, and/or the use of natural therapeutic substances (such as, for example, hypericum which represents a substance used in PPD, but not without side effects).

Note that, in most cases, PPD occurs in the puerperal woman between 4 and 6 weeks after delivery (Melissa M Batt et al. “Is Postpartum Depression Different From Depression Occurring Outside of the Perinatal Period?A Review of the Evidence”. Focus (Am Psychiatr Publ) 2020 April; 18(2):106-119; Clare C A, Yeh J. Postpartum depression in special populations: a review. Obstet Gynecol Surv. 2012 May; 67(5):313-23. doi: 10.1097/OGX.0b013e318259b52. PMID: 22624779).

Note that, preferably, the composition is suitable for the prevention of PPD in
pregnant women, preferably in pregnant women between the 35^{th} and the 40^{th} week of pregnancy, or
puerperal women in the neonatal period comprised between 0 and 6 weeks, preferably between 2 and 6 weeks, preferably between 2 and 4 weeks after delivery.

According to a preferred embodiment, the composition can be taken by the pregnant woman, in the last weeks of pregnancy, maintaining the therapy also in the postnatal period (also called neonatal).

Preferably, the composition is useful in the treatment of PPD in puerperal women. Still, the composition is useful for the treatment of puerperal women in the neonatal period comprised between 0 weeks and 24 months, preferably comprised between 0 weeks and 18 months, preferably comprised between 0 weeks and 12 months, preferably comprised between 0 weeks and 9 months, preferably comprised between 0 weeks and 6 months, preferably comprised between 0 weeks and 20 weeks, preferably comprised between 0 weeks and 16 weeks, preferably comprised between 0 weeks and 12 weeks, preferably comprised between 0 weeks and 8 weeks, preferably comprised between 2 weeks and 8 weeks, preferably comprised between 4 weeks and 8 weeks.

Note that, preferably, PPD is characterized by a symptomatology chosen from the group consisting of: rapid mood swings, irritability, anxiety or panic attacks, decrease of concentration, crying crisis (together known as “baby blues”); appetite disorders (such as loss of appetite or hyperphagia); sleep disorders (such as insomnia or hypersomnia); insomnia even when the child sleeps; psychomotor agitation or slowdown; fatigue/tiredness; suicidal thoughts; depressed mood; loss of libido; headaches and myalgias; excessive concerns or disinterest in the baby; feeling of uselessness or inadequacy as a mother; sense of guilt; fear of harming the baby and combinations thereof.

### EXAMPLES

### 1. Capsules Comprising the Composition of the Invention.

For illustrative and non-limiting purposes, an embodiment of the composition object of the invention is shown below.

| Components | For 1 capsule | |
|---|---|---|
| ASHWAGANDHA (WITHANIA SOMNIFERA | 0.15 | g |
| (L.) DUNAL, ROOTS) D.E. TIT 5% | | |
| WITHANOLIDES | | |
| LAVANDER (Lavandula angustifolia) | 0.1 | g |
| ESSENTIAL OIL MICROENCAPSULATED | | |
| LEMON BALM (MELISSA OFFICINALIS L. | 0.1 | g |
| LEAVES) D.E. TIT. 12% ROSMARINIC ACID | | |
| CELLULOSE - E460(i) | 0.031 | g |
| CHOLECALCIFEROL (VITAMIN D) 0.25% | 0.02 g/0.05 g | |
| MAGNESIUM SALT OF FATTY ACIDS - E470b | 0.002 | g |
| SILICON DIOXIDE - E551 | 0.002 | g |
| VEGETAL CAPSULE (HYDROXYPROPYL | 1 | pc |

METHYL CELLULOSE - E464)

2. Clinical Data: Evaluation of the Efficacy of a Preparation Based on Natural Substances Such as Ashwagandha (150 mg), Lavender O.E. (100 mg) Melissa officinalis (100 mg) with Addition of Vitamin D3 (50 μg) in the Treatment of Postpartum Depression.

The study aims to evaluate the efficacy and the tolerability of a new product consisting of natural substances capable of counteracting and preventing the onset of postpartum depression.

Fifty women aged 18 to 40 years were recruited in the first week after a natural birth or after caesarean section, suffering from postpartum depression diagnosed by self-assessment through the completion of the EPDS questionnaire (Edinburgh Postnatal Depression Scale). EPDS is a self-assessment questionnaire, consisting of 10 items that investigate the presence and the intensity of depressive symptoms with reference to: anhedonia, sense of guilt, anxiety, fear or panic, sadness and crying, sense of failure, sleep difficulties, thoughts of hurting oneself. The woman can choose between four answers, whose score (0-3) varies in an increasing manner by the severity of the symptom investigated. The minimum and maximum scores are 0 and 30.

The woman defines herself as suffering from PPD when the score is >12.5.

The women who joined the study were treated for 12 weeks, with a monotherapy comprising a daily administration of a solid oral composition (capsule) in accordance with Example 1.

After 6 weeks (T1) and 12 weeks (T2) from the start of the treatment (TO) with this new phytotherapeutic (nutraceutical) product, the patients filled out again the questionnaire.

### 2.1 EPDS Questionnaire

Below is the content in English of the EPDS form used for the purposes of the clinical study in question:

Edinburgh Postnatal Depression Scale (EPDS)

| | |
|---|---|
| Name: | Address: |
| Your birth date: | |
| Your child's date of birth: | Telephone number: |

Since you were pregnant and recently had a baby, we would like to know how you are
feeling. Please select the answer that comes closest to your mood IN THE LAST 7 DAYS,
not the one that comes closest to today's mood.
Below is an example, already completed.

| | |
|---|---|
| I felt happy: | This would mean: “I felt happy most of the |
| [symbol] yes, all the time | time” in the past week. |
| [symbol] yes, most of the time | Please complete the other questions in the |
| [symbol] no, not very frequently | same way. |
| [symbol] no, not at all. | |

In the last 7 days:

| | |
|---|---|
| 1. I was able to smile and see the funny side | 6. I was overwhelmed by things. |
| of things. | [symbol] Yes, most of the time I haven't been able |
| [symbol] As much as I have always done | to handle myself at all. |
| [symbol] Not so much now | [symbol] Yes, I have sometimes been unable to |
| [symbol] Definitely not so much now | handle myself as usual. |
| [symbol] Not at all | [symbol] No, most of the time I handled myself |
| | pretty well. |
| | [symbol] No, I was able to handle myself as much |
| | as I used to. |
| 2. I was looking forward to the upcoming | 7. I was so unhappy that I couldn't sleep. |
| things with joy. | [symbol] Yes, most of the time |
| [symbol] As much as I have always done | [symbol] Yes, sometimes |
| [symbol] Less than before | [symbol] Not very often |
| [symbol] Definitely less than before | [symbol] No, not at all |
| [symbol] Almost nothing | |
| 3. I berated myself for no reason when things | 8. I felt sad and down. |
| went wrong. | [symbol] Yes, most of the time |
| [symbol] Yes, most of the time | [symbol] Yes, sometimes |
| [symbol] Yes, sometimes | [symbol] Not very often |
| [symbol] Not very often | [symbol] No, not at all |
| [symbol] Never | |
| 4. I was nervous and worried for no real | 9. I felt so sad that I burst into tears. |
| reason. | [symbol] Yes, most of the time |
| [symbol] Not at all | [symbol] Yes, sometimes |
| [symbol] Hardly ever | [symbol] Occasionally only |
| [symbol] Yes, sometimes | [symbol] No, never |
| [symbol] Yes, frequently | |
| 5. I felt scared or panicked for no real | 10. The thought of hurting myself came to my |
| reason. | mind. |
| [symbol] Yes, a lot | [symbol] Yes, quite often |
| [symbol] Yes, sometimes | [symbol] Sometimes |
| [symbol] No, not much | [symbol] Hardly ever |
| [symbol] No, not at all | [symbol] Never |

EPDS questionnaire (10 items with a score from 0 to 3 indicating the well-being in descending order); women who score between 0 and 12 are not depressed, those who score between 13 and 30 have a possible or certain diagnosis of postpartum depression.

### 2.2 Results

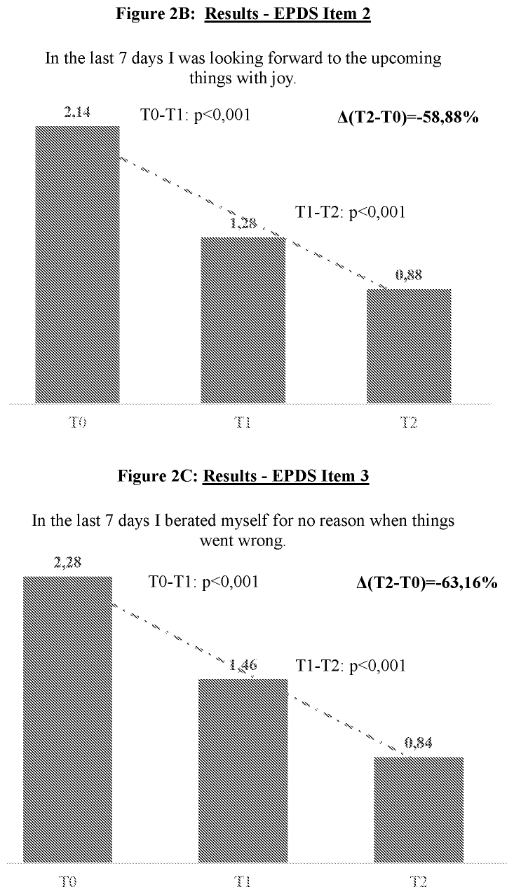

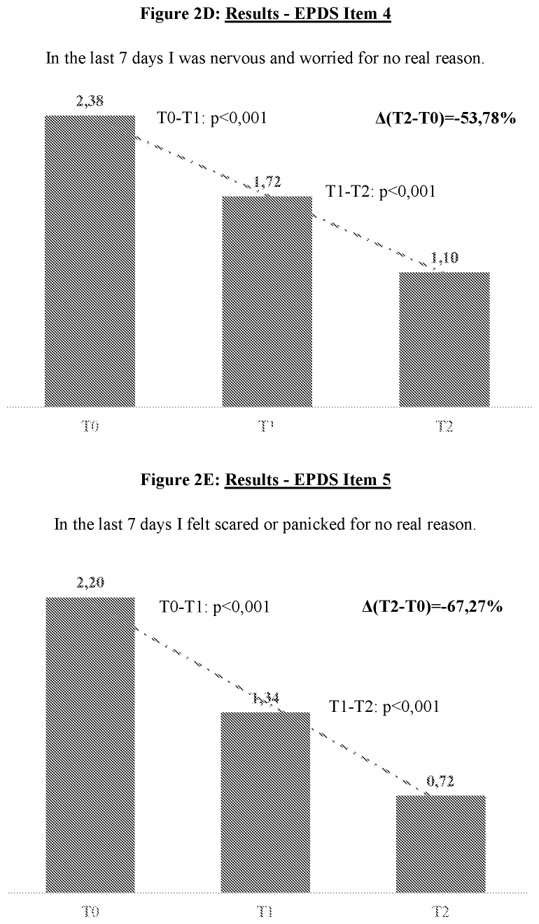

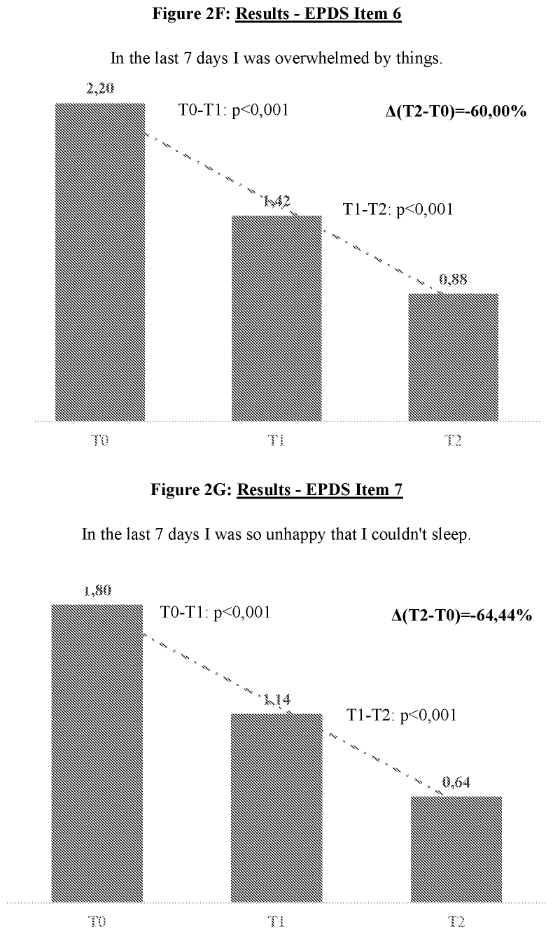

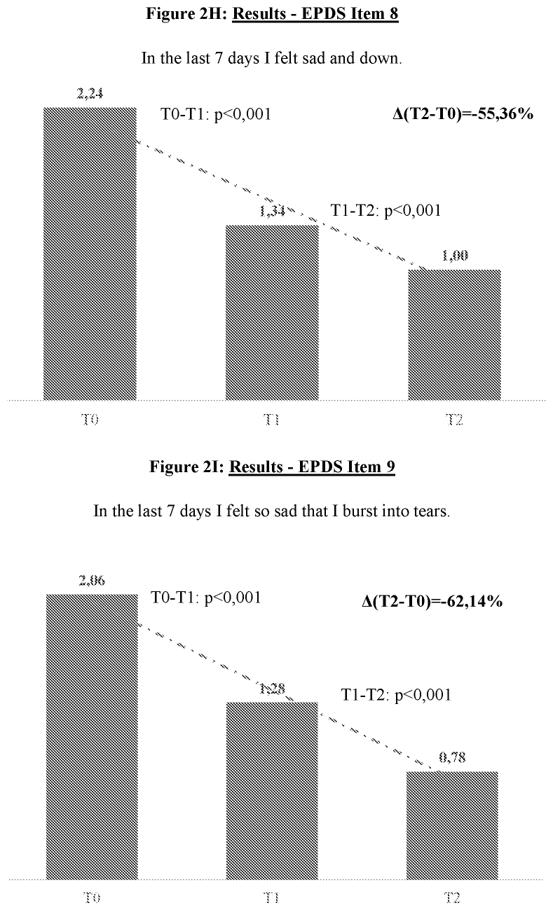

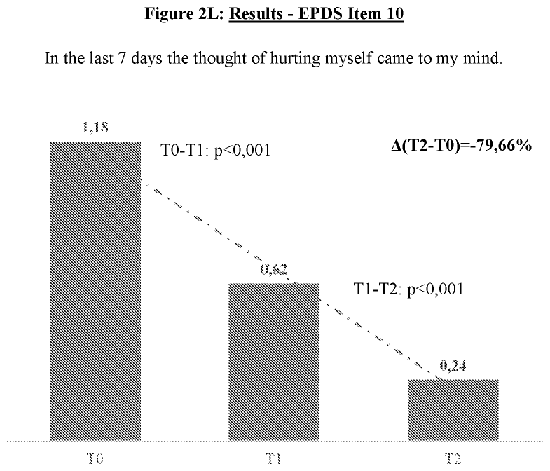

FIGS. 2A-2L show, for each item of the questionnaire, the arithmetic mean of the score given by each patient from the start of the therapy to the end of the study (T0, T1, T2).

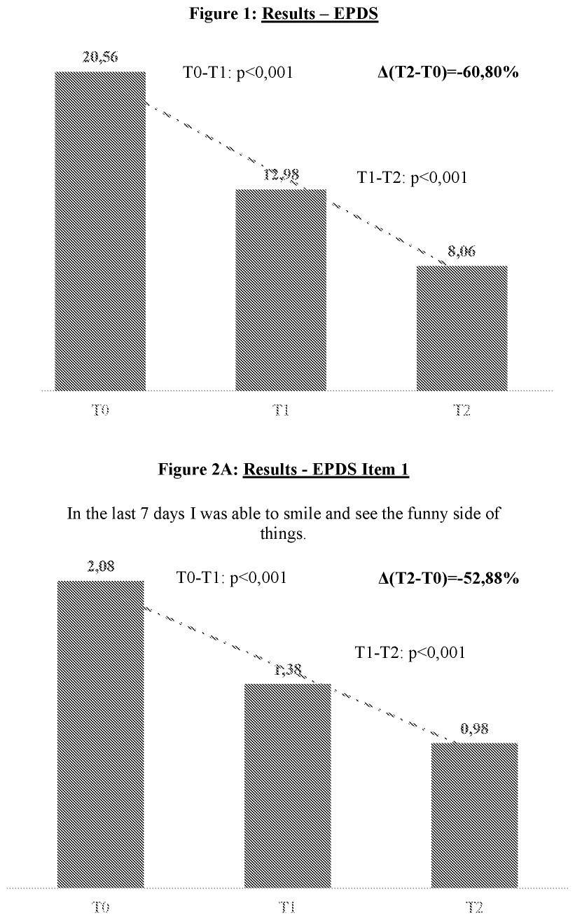

FIG. 1 shows the means of the sum of the scores recorded by each patient, for each item of the EPDS questionnaire, from the start of therapy to the end of the study (T0, T1, T2).

In the Table 1 below, the same values that can be found in FIG. 1 (sum of the scores) and in FIGS. 2A-2L (arithmetic mean of the score of each patient) together with the indication of the relative standard deviation (SD) are indicated.

TABLE 1
Scores and related standard deviations obtained by processing
the data collected at the end of the clinical study: the values
indicate both the mean scores obtained for each item of the
questionnaire (Item 1-10), and the sum of the mean scores
recorded for each item of the questionnaire (Sum).

| | | |
|---|---|---|
| T0 | T1 | T2 |

| Reference | Value | SD | Value | SD | Value | SD |
|---|---|---|---|---|---|---|
| Sum | 20.56 | ±3.11 | 12.98 | +2.96 | 8.06 | +2.77 |
| Item 1 | 2.08 | ±0.52 | 1.38 | ±0.58 | 0.98 | ±0.42 |
| Item 2 | 2.14 | +0.63 | 1.28 | ±0.49 | 0.88 | +0.55 |
| Item 3 | 2.28 | +0.57 | 1.46 | +0.61 | 0.84 | ±0.50 |
| Item 4 | 2.38 | ±0.72 | 1.72 | ±0.60 | 1.10 | ±0.58 |
| Item 5 | 2.20 | +0.49 | 1.34 | ±0.62 | 0.72 | ±0.57 |
| Item 6 | 2.20 | ±0.53 | 1.42 | ±0.53 | 0.88 | ±0.52 |
| Item 7 | 1.80 | +0.57 | 1.14 | ±0.53 | 0.64 | ±0.52 |
| Item 8 | 2.24 | ±0.51 | 1.34 | +0.55 | 1.00 | ±0.45 |
| Item 9 | 2.06 | ±0.61 | 1.28 | ±0.69 | 0.78 | ±0.58 |
| Item 10 | 1.18 | +0.99 | 0.62 | ±0.75 | 0.24 | +0.47 |

For the purpose of comparing the mean of the scores in the two times TO-T1 and T1-T2, a t-test (paired, two-tailed t-test) was applied; for the evaluation of the significance of the difference of the scores found at T1 with respect to T0, and of the scores found at T2 with respect to T1, the p-value was also calculated, with a threshold value a of 0.001.

As can be seen from FIGS. 2A-2L, for each of the items of the EPDS questionnaire there is a reduction in score as time progresses.

The statistical analysis was carried out on the sum of the mean scores (FIG. 1), to evaluate whether the treatment resulted in a significant overall improvement in the sum of the mean scores, and therefore of the patients under examination; both on the scores of the individual items constituting the EPDS questionnaire (i.e. for each question), to evaluate whether the treatment resulted in a significant improvement in terms of the presence and intensity of the depressive symptoms of interest (anhedonia, sense of guilt, anxiety, fear or panic, sadness and crying, sense of failure, sleep difficulties, thoughts of hurting oneself).

Considering the minimum score of 12.5 for the diagnosis of PPD by means of EPDS, it can be noted from FIG. 1 that the sum of the mean scores drops significantly, from a value of 20.56 to a value of 12.98, already 6 weeks after the start of the treatment (TO-T1: p<0.001), thus approaching the threshold value that allows to discriminate between the presence or not of the pathology. The score drops significantly below this threshold after 12 weeks from the start of the treatment. Note that the reduction recorded between time T2 and time T1 was also significant (T2-T1: p<0.001). Overall (FIG. 1), the sum of the mean scores recorded at time T2 was reduced by about 61% compared to the mean score recorded at time TO, and by about 38% compared to the mean score recorded at time T1.

For each test item (i.e. for each question), the score reduction recorded 6 weeks after the start of the treatment (T0-T1) and the score reduction recorded 6 weeks after half of the treatment (T1-T2) were significant (p<0.001). It can therefore be concluded that the composition object of the invention was effective in the treatment of postpartum depression.

1. A method for the prevention and/or the treatment of postpartum depression (PPD) said method comprising
administering a composition comprising, as active ingredients:
dry extract of Withania somnifera (L.),
dry extract of Melissa officinalis (L.), and
microencapsulated essential oil of Lavandula angustifolia.

2. The method according to claim 1, wherein the composition is an adjuvant of pharmacological therapies.

3. The method according to claim 1, for in the prevention of PPD in
pregnant women, or
puerperal women in the neonatal period comprised between 0 and 6 weeks after delivery.

4. The method according to claim 1, for the treatment of PPD of puerperal women.

5. The method according to claim 4, for the treatment of PPD of puerperal women in the neonatal period comprised between 0 weeks and 24 months.

6. The method according to claim 1, wherein PPD is characterized by a symptomatology selected from the group consisting of: rapid mood swings, irritability, anxiety or panic attacks, decrease of concentration, crying crisis; appetite disorders; sleep disorders or insomnia even when the child sleeps; psychomotor agitation or slowdown; fatigue/tiredness; suicidal thoughts; depressed mood; loss of libido; headaches and myalgias; excessive concerns or disinterest in the baby; feeling of uselessness or inadequacy as a mother; sense of guilt; fear of harming the baby and combinations thereof.

7. The method according to claim 1, wherein the composition comprises
dry extract of Withania somnifera in a concentration comprised between 30% and 40% (w/w),
dry extract of Melissa officinalis, in a concentration comprised between 20% and 30% (w/w),
microencapsulated essential oil of Lavandula angustifolia, in concentration comprised between 20% and 30% (w/w),
per administration unit.

8. The method according to claim 1, wherein the composition comprises, as a further active ingredient vitamin D3.

9. The method according to claim 1, wherein the composition is administered daily by a dosage of 1 or 2 administration units.

10. The method according to claim 1, wherein the composition is in solid form.

11. The method according to claim 3, wherein said pregnant women are between the 35^{th} and 40^{th} week of pregnancy.

12. The method according to claim 8, wherein said vitamin D3 is in concentration comprised between 3% and 8% (w/w) per administration unit.

13. The method according to claim 10, wherein said solid form is the form of tablet, capsule or powder.
